(19) United States
(12) Patent Application Publication
(10) Pub. No.: US 2017/0043307 A1
(43) Pub. Date: Feb. 16, 2017

(54) METHOD FOR PREPARING MEDICAL/CHEMICAL SOLUTION AND MEDICAL/CHEMICAL SOLUTION PREPARATION SYSTEM

(71) Applicant: Kabushiki Kaisha Yaskawa Denki, Kitakyushu-shi (JP)

(72) Inventors: Shigeji TASAKA, Kitakyushu-shi (JP); Teruhiro KURIHARA, Kitakyushu-shi (JP)

(73) Assignee: Kabushiki Kaisha Yaskawa Denki, Kitakyushu-shi (JP)

(21) Appl. No.: 15/226,318

(22) Filed: Aug. 2, 2016

(30) Foreign Application Priority Data
Aug. 11, 2015 (JP) 2015-159247

(51) Int. Cl. B01F 15/00; A61K 9/08; B25J 9/16; B25J 9/00; B25J 15/08; A61J 3/00; B01F 13/00

(52) U.S. Cl. CPC B01F 15/00279; A61J 3/00; A61K 9/08; B01F 13/003; B25J 9/0087; B25J 15/08; B25J 9/1612; B25J 9/161; B01F 2215/0032; Y10S 901/02

(57) ABSTRACT

A method is for preparing a medical/chemical solution that includes a mixture of a medical/chemical substance and a solution. The method includes, after the solution has been injected from a syringe into a container containing the medical/chemical substance, shaking the container using a robot with the container held by a hand mounted on one end of an arm of the robot.

### CROSS-REFERENCE TO RELATED APPLICATIONS

The present application claims priority under 35 U.S.C. §119 to Japanese Patent Application No. 2015-159247, filed Aug. 11, 2015. The contents of this application are incorporated herein by reference in their entirety.

### BACKGROUND

Field of the Invention

The embodiments disclosed herein relate to a method for preparing a medical/chemical solution and to a medical/chemical solution preparation system.

Discussion of the Background

Medical/chemical solution preparation systems prepare medical/chemical solutions by mixing medical/chemical substances with solutions.

### SUMMARY

According to one aspect of the present disclosure, a method is for preparing a medical/chemical solution that includes a mixture of a medical/chemical substance and a solution. The method includes, after the solution has been injected from a syringe into a container containing the medical/chemical substance, shaking the container using a robot with the container held by a hand mounted on one end of an arm of the robot.

According to another aspect of the present disclosure, a medical/chemical solution preparation system includes a robot and a controller. The robot includes an arm including a hand mounted on one end of the arm. The controller is configured to control the robot to prepare a mixture of a medical/chemical substance and a solution. The controller includes a first controller configured to control the robot to, after the solution has been injected from a syringe into a container containing the medical/chemical substance, shake the container with the container held by the hand.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 is a schematic illustrating an exemplary configuration of a medical/chemical solution preparation system according to an embodiment;

FIG. 2 illustrates an exemplary configuration of a robot according to the embodiment;

FIG. 3 illustrates an exemplary configuration of a syringe driver according to the embodiment;

FIG. 4 illustrates an example of how a syringe and a container are held by the robot and the syringe driver according to the embodiment;

FIG. 5 illustrates an exemplary configuration of a stirrer according to the embodiment;

FIG. 6 is a diagram illustrating an exemplary configuration of a controller according to the embodiment;

FIG. 7 is a diagram illustrating an exemplary functional configuration of the controller according to the embodiment;

FIG. 8 illustrates an exemplary operation to shake the container using the robot according to the embodiment; and

FIG. 9 is a flowchart of an exemplary method according to the embodiment for preparing a medical/chemical solution.

### DESCRIPTION OF THE EMBODIMENTS

The embodiments will now be described with reference to the accompanying drawings, wherein like reference numerals designate corresponding or identical elements throughout the various drawings.

### Medical/chemical Solution Preparation System

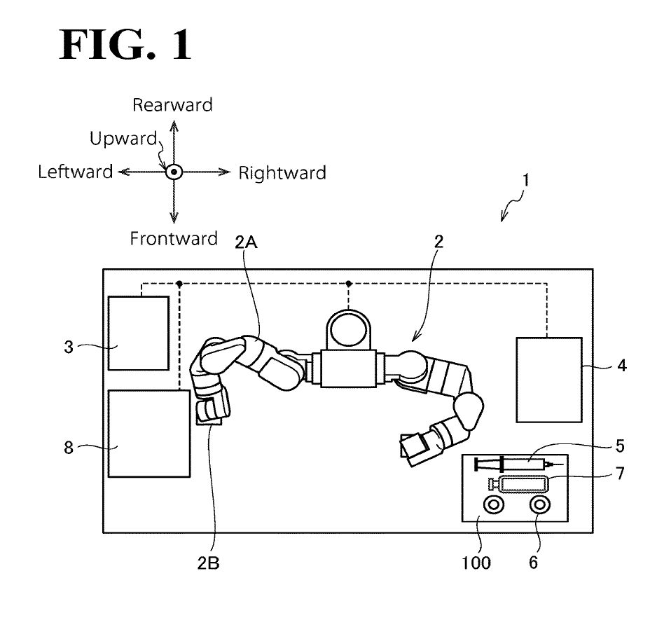

By referring to FIGS. 1 to 9, a schematic configuration of a medical/chemical solution preparation system 1 according to this embodiment will be described. For convenience of description of the medical/chemical solution preparation system 1 and other and related elements, directions indicated by “upward”, “downward”, “rightward”, “leftward”, “frontward”, and “rearward” are indicated in some of FIGS. 1 to 9. It should be noted, however, that these directions may vary depending on where to install the medical/chemical solution preparation system 1 and other and related elements, and should not be construed as limiting their positions in relation to each other.

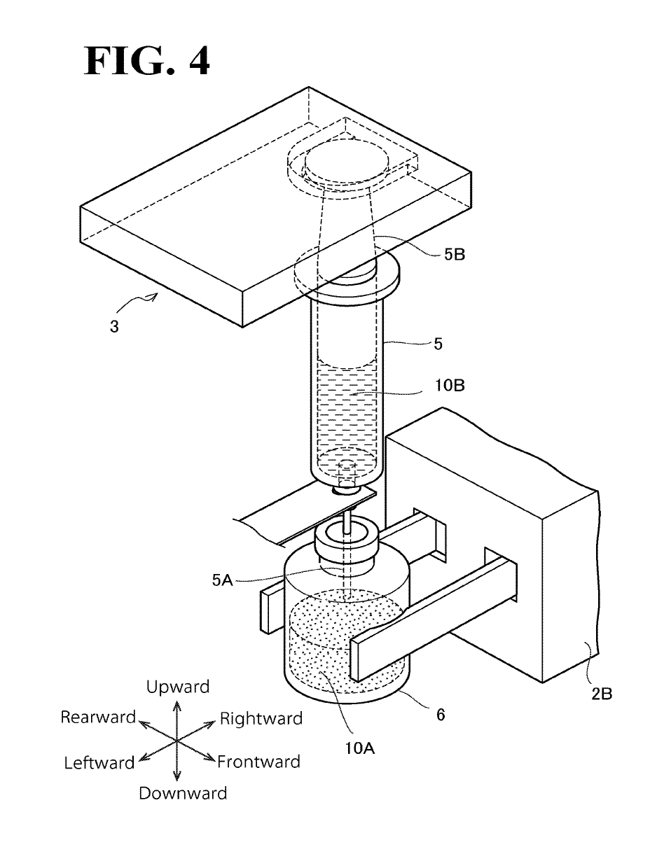

The medical/chemical solution preparation system 1 according to this embodiment mixes a medical/chemical substance 10A and a solution 10B with each other (see FIG. 4, described later). The medical/chemical substance 10A is hermetically sealed in a container 6 (a non-limiting example of is a vial) in liquid form or powdery form. The solution 10B is hermetically sealed in a solution bag 7. Examples of the solution 10B include, but are not limited to, a saline solution and a dextrose solution.

As illustrated in FIG. 1, the medical/chemical solution preparation system 1 according to this embodiment includes a robot 2, a syringe driver 3, a controller 4, and a stirrer 8. Also as illustrated in FIG. 1, while the medical/chemical solution preparation system 1 according to this embodiment is not in its mixture operation, a syringe 5, the container 6, and the solution bag 7 are placed on a tray 100.

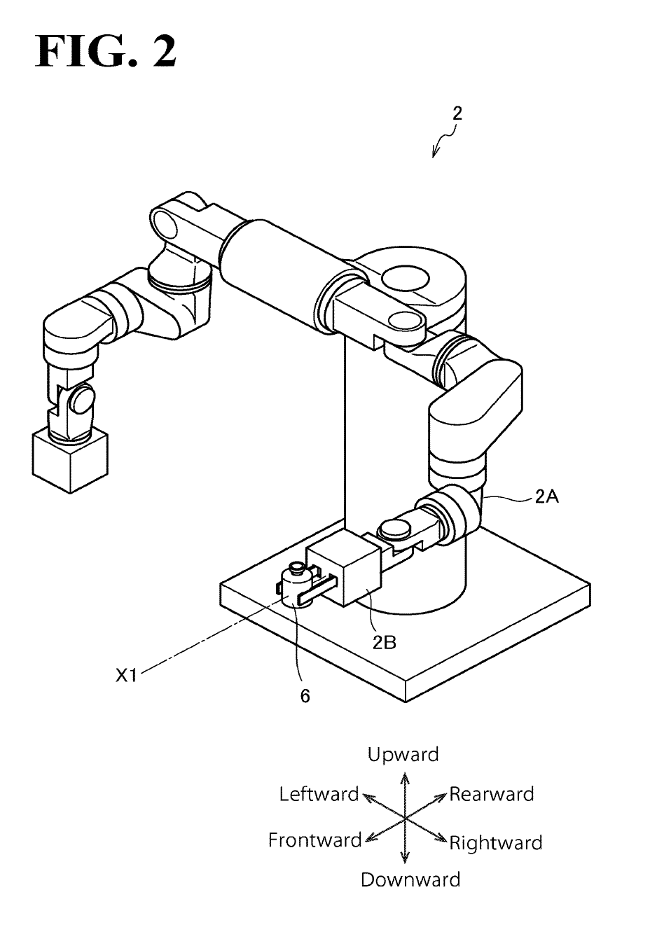

As illustrated in FIGS. 1 and 2, the robot 2 includes an the arm 2A. The arm 2A is attached with a hand 2B at one end (distal end) of the arm 2A. The hand 2B is capable of holding at least one of the syringe 5 and the container 6. In this embodiment, the robot 2 is a two-arm robot, which is equipped with two multi-articular arms 2A.

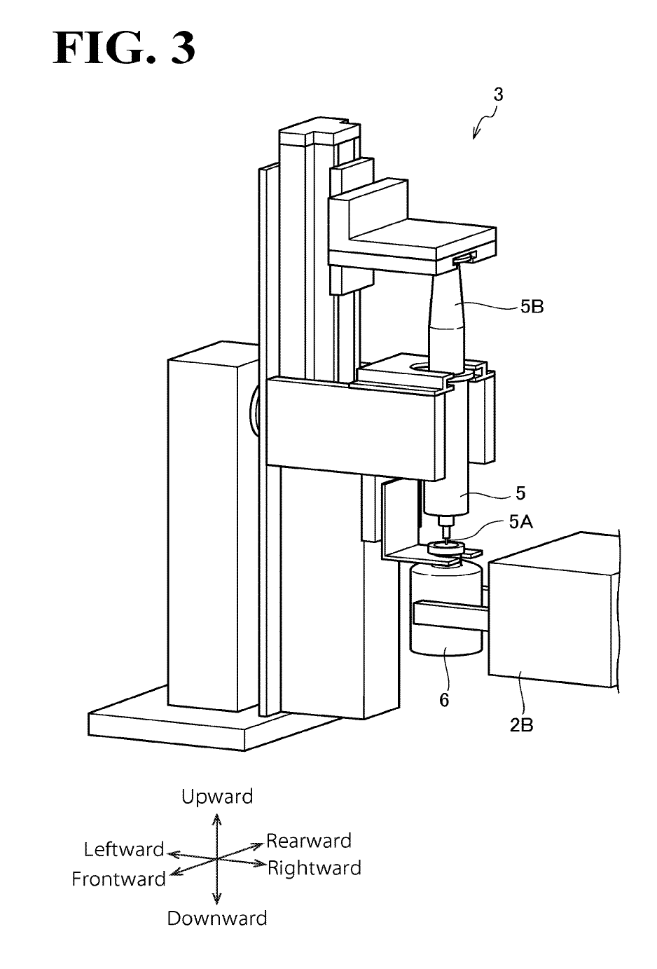

As illustrated in FIG. 3, the syringe driver 3 is capable of holding the syringe 5 and the container 6 and driving a plunger 5B of the syringe 5.

In another possible embodiment of the medical/chemical solution preparation system 1, the syringe driver 3 may be capable of holding either the syringe 5 or the container 6. In this case, the syringe driver 3 cooperates with the robot 2 to drive the plunger 5B of the syringe 5.

In still another possible embodiment of the medical/chemical solution preparation system 1, the syringe 5 may be held by the syringe driver 3 and the container 6 may be held by the robot 2, or the syringe 5 may be held by the robot 2 and the container 6 may be held by the syringe driver 3. In this case, the robot 2 and the syringe driver 3 cooperate with each other to drive the plunger 5B of the syringe 5 so as to transfer the solution 10B contained in the syringe 5 into the container 6.

In still another possible embodiment of the medical/chemical solution preparation system 1, both the syringe 5 and the container 6 may be held by the syringe driver 3 (see FIG. 4), or both the syringe 5 and the container 6 may be held by the robot 2. In this case, the robot 2 or the syringe driver 3 drives the plunger 5B of the syringe 5 so as to transfer the solution 10B contained in the syringe 5 into the container 6.

In the medical/chemical solution preparation system 1 according to this embodiment, the syringe 5 includes a needle 5A and the plunger 5B. The needle 5A and the plunger 5B are used in the transfer of the solution 10B. In still another possible embodiment of the medical/chemical solution preparation system 1, the syringe 5 may come in a variety of diameters or lengths, among which a suitable syringe 5 may be selected according to the kind, volume, or any other parameter of the solution 10B to be transferred. In the embodiment illustrated in FIG. 1, one kind of syringe 5 is used for convenience of description, although two or more kinds of syringes 5 may be used.

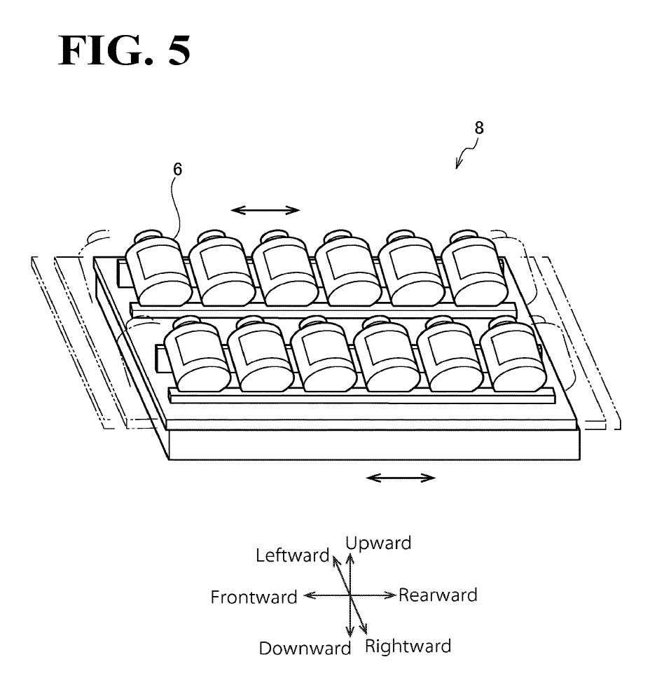

As illustrated in FIG. 5, the stirrer 8 stirs one container 6 or a plurality of the containers 6 placed in the stirrer 8. In the medical/chemical solution preparation system 1 according to this embodiment, the stirrer 8 moves in the frontward and rearward directions to stir one container 6 or a plurality of the containers 6 placed in the stirrer 8.

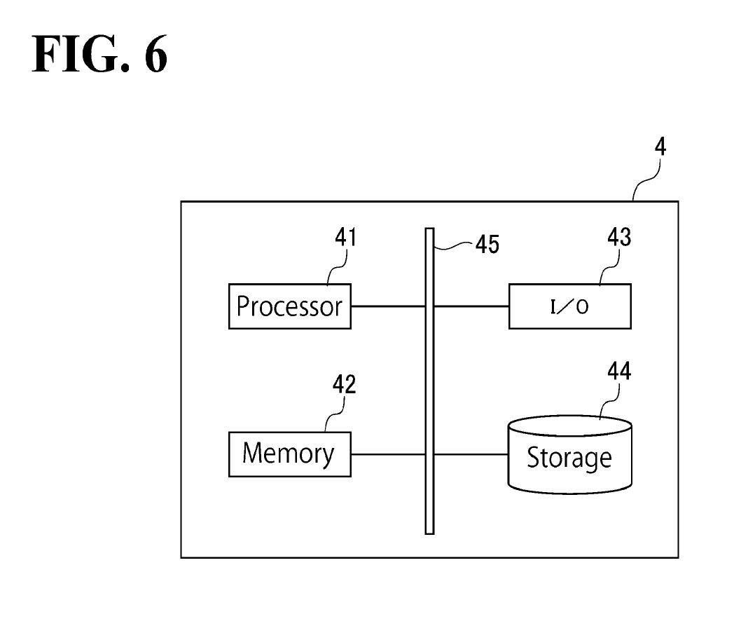

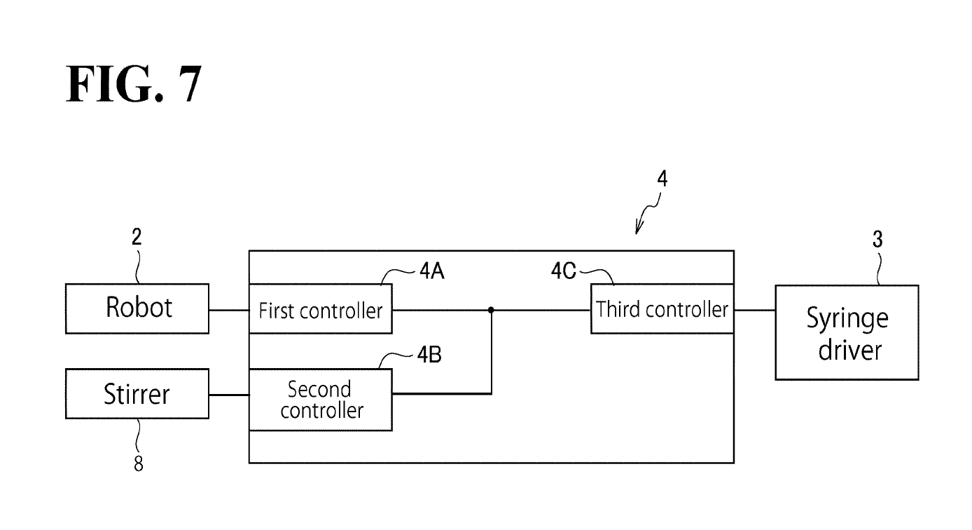

As illustrated in FIG. 1, the controller 4 controls the robot 2, the syringe driver 3, and the stirrer 8. As illustrated in FIG. 6, the controller 4 includes a processor 41, a memory 42, an input/output section (I/O) 43, a storage 44, and a bus 45. The bus 45 connects these elements to each other. The processor 41 cooperates with at least one of the memory 42 and the storage 44 to execute a program, and uses a result of executing the program as a basis for inputting and outputting data through the input/output section 43. In this manner, the controller 4 implements various functions. FIG. 7 illustrates these functions in the form of imaginary blocks (hereinafter referred to as functional blocks).

As illustrated in FIG. 7, the functional blocks of the controller 4 are a first controller 4A, a second controller 4B, and a third controller 4C. The first controller 4A controls the robot 2, the second controller 4B controls the stirrer 8, and the third controller 4C controls the syringe driver 3.

The first controller 4A and the third controller 4C respectively control the robot 2 and the syringe driver 3 to transfer the solution 10B from the solution bag 7 into the syringe 5. In this case, the robot 2 uses the hand 2B to place the syringe 5 and the solution bag 7 in the syringe driver 3, and the robot 2 and the syringe driver 3 cooperate with each other to transfer the solution 10B from the solution bag 7 into the syringe 5.

In another possible embodiment, the first controller 4A may control the robot 2 alone to transfer the solution 10B from the solution bag 7 into the syringe 5. In this case, the robot 2 may transfer the solution 10B from the solution bag 7 into the syringe 5 with the hand 2B securing the syringe 5 and the solution bag 7 to a predetermined table (not illustrated).

The first controller 4A and the third controller 4C respectively control the robot 2 and the syringe driver 3 to inject the solution 10B into the container 6 from the syringe 5. Specifically, the hand 2B at the distal end of one arm 2A of the robot 2 holds the container 6 and the hand 2B at the distal end of the other arm 2A places the syringe 5 in the syringe driver 3. Then, the robot 2 and the syringe driver 3 cooperate with each other to transfer the solution 10B from the syringe 5 into the container 6. In another possible embodiment, the robot 2 may, using the hand 2B, place the syringe 5 and the container 6 in the syringe driver 3, and the syringe driver 3 may transfer the solution 10B from the syringe 5 into the container 6.

In still another possible embodiment, the first controller 4A may control the robot 2 alone to inject the solution 10B into the container 6 from the syringe 5. In this case, the robot 2 may transfer the solution 10B from the syringe 5 into the container 6 with the hand 2B securing the syringe 5 and the container 6 to a predetermined table (not illustrated).

After the solution 10B has been injected into the container 6 from the syringe 5, the first controller 4A controls the robot 2 to shake the container 6 with the container 6 being held by the hand 2B.

In a possible embodiment where the solution 10B is transferred into the container 6 with the container 6 being held by the syringe driver 3, the first controller 4A may control the robot 2 to hold the container 6 using the hand 2B and then shake the container 6.

This configuration enables the container 6 to be shaken immediately after the solution 10B is injected into the container 6 containing the medical/chemical substance 10A This, in turn, prevents the medical/chemical substance 10A and the solution 10B from being solidified in the container 6. For example, some medical/chemical substances such as adriacin may possibly become solidified into a jelly form unless stirred immediately after introduction of the solution 10B (which is a phenomenon referred to as stacking). Use of the above-described configuration eliminates or minimizes this phenomenon.

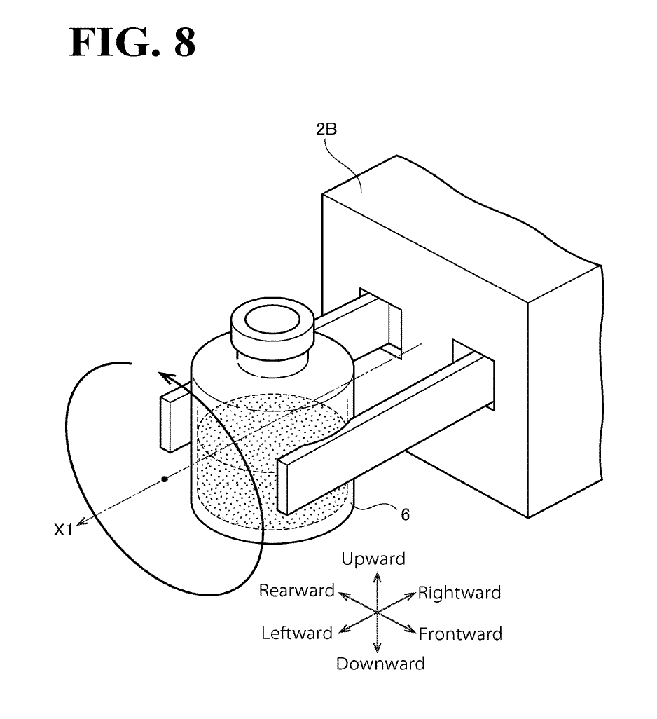

As illustrated in FIG. 8, the first controller 4A controls the robot 2 to turn the hand 2B upside down about wrist axis X1 of the arm 2A, so as to shake the container 6. This configuration enables the medical/chemical substance 10A and the solution 10B to be more readily mixed together in the container 6 by being turned upside down.

After a plurality of the containers 6 have been shaken with each of the plurality of the containers 6 held by the hand 2B and after the plurality of the containers 6 have been placed in the stirrer 8, the second controller 4B operates the stirrer 8. Specifically, the first controller 4A controls the robot 2 to place each container 6 in the stirrer 8. This configuration ensures reliability in mixing the medical/chemical substance 10A and the solution 10B together in the stirrer 8 after the container 6 has been shaken by the robot 2.

Based on the kind of the medical/chemical substance 10A (for example, a kind of the medical/chemical substance 10A with particular viscosity), the first controller 4A determines whether to control the robot 2 to shake the container 6. This configuration enables the robot 2 to shake the container 6 only when it is likely considering the viscosity or another property of the medical/chemical substance 10A that the medical/chemical substance 10A and the solution 10B become solidified after the medical/chemical substance 10A and the solution 10B are mixed together. Thus, unnecessary movement of the robot 2 is eliminated or minimized.

### Method for Preparing Medical/chemical Solution

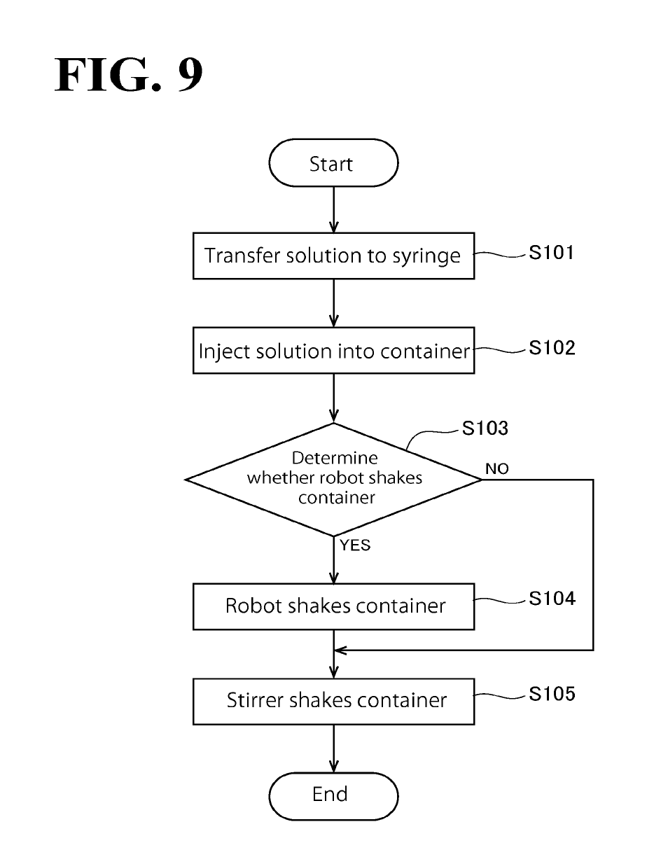

By referring to FIG. 9, description will be made with regard to an exemplary method for preparing a medical/chemical solution performed by the controller 4 using the robot 2, the syringe driver 3, and the stirrer 8. The method for preparing a medical/chemical solution according to this embodiment is a method for mixing the medical/chemical substance 10A and the solution 10B together.

As illustrated in FIG. 9, at step S101, the controller 4 causes the solution 10B to be transferred from the solution bag 7 into the syringe 5. At step S102, the controller 4 causes the plunger 5B of the syringe 5 to be pressed so as to inject the solution 10B into the container 6.

At step S103, based on, for example, the kind of the medical/chemical substance 10A, the controller 4 determines whether to control the robot 2 to shake the container 6. When the controller 4 determines to control the robot 2 to shake the container 6, the controller 4 performs the operation at step S104. When the controller 4 determines not to control the robot 2 to shake the container 6, the controller 4 performs the operation at step S105.

At step S104, the controller 4 controls the robot 2 to shake the container 6 by, for example, turning the hand 2B upside down about wrist axis X1 of the arm 2A.

At step S105, after the robot 2 has placed a plurality of the containers 6 in the stirrer 8, the controller 4 operates the stirrer 8. Here, the plurality of the containers 6 placed in the stirrer 8 may include those containers 6 that have been shaken by the robot 2 and those containers 6 that are not shaken by the robot 2.

The medical/chemical substance prepare system 1 according to this embodiment and the method according to this embodiment for preparing a medical/chemical solution widen the variety of medical/chemical substances 10A that can be mixed.

Obviously, numerous modifications and variations of the present disclosure are possible in light of the above teachings. It is therefore to be understood that within the scope of the appended claims, the present disclosure may be practiced otherwise than as specifically described herein.

What is claimed as new and desired to be secured by Letters Patent of the United States is:

1. A method for preparing a medical/chemical solution, the medical/chemical solution comprising a mixture of a medical/chemical substance and a solution, the method comprising, after the solution has been injected from a syringe into a container containing the medical/chemical substance, shaking the container using a robot with the container held by a hand mounted on one end of an arm of the robot.

2. The method according to claim 1, wherein the shaking step comprises turning the hand upside down about a wrist axis of the arm, so as to shake the container.

3. The method according to claim 1, further comprising:
after the shaking step, placing the container in a stirrer using the robot; and
after a plurality of the containers have been placed in the stirrer, operating the stirrer.

4. The method according to claim 1, further comprising determining, based on a kind of the medical/chemical substance, whether to shake the container with the container held by the hand.

5. A medical/chemical solution preparation system comprising:
a robot comprising an arm comprising a hand mounted on one end of the arm; and
a controller configured to control the robot to prepare a mixture of a medical/chemical substance and a solution, the controller comprising a first controller configured to control the robot to, after the solution has been injected from a syringe into a container containing the medical/chemical substance, shake the container with the container held by the hand.

6. The medical/chemical solution preparation system according to claim 5, wherein the first controller is configured to control the robot to turn the hand upside down about a wrist axis of the arm, so as to shake the container.

7. The medical/chemical solution preparation system according to claim 5, wherein the controller comprises a second controller configured to operate a stirrer after a plurality of the containers have been shaken with each of the plurality of the containers held by the hand and after the plurality of the containers have been placed in the stirrer.

8. The medical/chemical solution preparation system according to claim 5, wherein the first controller is configured to determine, based on a kind of the medical/chemical substance, whether to shake the container with the container held by the hand.

9. The method according to claim 2, further comprising:
after the shaking step, placing the container in a stirrer using the robot; and
after a plurality of the containers have been placed in the stirrer, operating the stirrer.

10. The method according to claim 2, further comprising determining, based on a kind of the medical/chemical substance, whether to shake the container with the container held by the hand.

11. The method according to claim 3, further comprising determining, based on a kind of the medical/chemical substance, whether to shake the container with the container held by the hand.

12. The method according to claim 9, further comprising determining, based on a kind of the medical/chemical substance, whether to shake the container with the container held by the hand.

13. The medical/chemical solution preparation system according to claim 6, wherein the controller comprises a second controller configured to operate a stirrer after a plurality of the containers have been shaken with each of the plurality of the containers held by the hand and after the plurality of the containers have been placed in the stirrer.

14. The medical/chemical solution preparation system according to claim 6, wherein the first controller is configured to determine, based on a kind of the medical/chemical substance, whether to shake the container with the container held by the hand.

15. The medical/chemical solution preparation system according to claim 7, wherein the first controller is configured to determine, based on a kind of the medical/chemical substance, whether to shake the container with the container held by the hand.

16. The medical/chemical solution preparation system according to claim 13, wherein the first controller is configured to determine, based on a kind of the medical/chemical substance, whether to shake the container with the container held by the hand.
